(19) United States
(12) Patent Application Publication
(10) Pub. No.: US 2013/0029422 A1
(43) Pub. Date: Jan. 31, 2013

(54) Composite Substrate for 3D Cell Culture

(71) Applicants: Vasiliy Nikolaevich Goral, Painted Post, NY (US); Yulong Hong, Painted Post, NY (US); Hui Su, Big Flats, NY (US); Jian Tan, Ithaca, NY (US)

(72) Inventors: Vasiliy Nikolaevich Goral, Painted Post, NY (US); Yulong Hong, Painted Post, NY (US); Hui Su, Big Flats, NY (US); Jian Tan, Ithaca, NY (US)

(21) Appl. No.: 13/190,589

(22) Filed: Jul. 26, 2011

(51) Int. Cl. C12N 5/071; C12N 1/20; C12N 1/16; C12N 5/04

(52) U.S. Cl. 435/402; 435/410; 435/252.1; 435/255.1

(57) ABSTRACT

A cell culture article comprises a substrate having a micro-structured surface and a thin hydrophobic elastomeric coating disposed on the substrate. The coating forms a micro-structured cell culture surface and is sufficiently thin to reduce absorption of hydrophobic molecules from an aqueous medium in contact with the coating, relative to articles fabricated entirely from the hydrophobic elastomer.

### FIELD

The present disclosure relates to cell culture, and more particularly to cell culture apparatuses having features to facilitate three-dimensional culture of cells.

### BACKGROUND

Cells cultured on flat cell culture surfaces often result in artificial two-dimensional sheets of cells that may have significantly different morphology and function from their in vivo counterparts. Cultured cells are crucial to modern drug discovery and development and are widely used for drug testing and screening. However, if results from such testing and screening are not indicative of responses from cells in vivo, the relevance of the results may be diminished. Cells in the human body experience three dimensional environments completely surrounded by other cells, membranes, fibrous layers, adhesion proteins, etc. Thus, substrates that prompt cultured cells to have in vivo-like morphology and function are desirable.

Cell culture articles having micro-structured surfaces, such as surfaces having patterned micro-cavities, that encourage three dimensional cell growth have been reported. Polydimethylsiloxane (PDMS) is a popular material used to develop cell culture apparatuses having micro-structured surfaces due to its biological compatibility, excellent optical transparency and gas permeability as well as its dynamic mechanical properties. For example, PDMS cell culture articles with interconnected microporous structures have been shown to promote in vivo-like three dimensional culture of breast cells and primary human hepatocytes, reproducing in vivo-like cell morphology and functionalities. Such PDMS surfaces may have significant implications for cancer research and drug discovery.

Although PDMS has desirable characteristics for cell culture, its value for cell-based application is undermined by its tendency to remove small hydrophobic molecules from aqueous phase. For example, PDMS-based cell culture articles have been shown to extract estrogen from cell culture medium during culture of breast cancer cells, significantly reducing the effects of estrogen on the cells. PDMS-based cell culture devices and substrates are also expected to face similar limitations in cytotoxicity studies of pharmaceutical compounds. By way of example, pharmaceutical compounds such as nefazadone, doxonibicin and paclitaxel were removed by PDMS from aqueous solution, and therefore their effects on cells could not be measured correctly in PDMS-based devices or substrates.

Cytotoxicity studies are important cell or tissue culture applications, and around 40% of marketed drugs are classified as “practically insoluble” or hydrophobic, and two thirds of synthesized drugs have low solubility. Therefore, the extraction of hydrophobic compounds from aqueous phase by PDMS or other hydrophobic elastomers should be addressed to develop practical culture devices and substrates based on this group of materials. Attempts to address this deficiency, such as pre-treating PDMS with serum or saturating PDMS with estrogen for 24 hours, have failed to prevent the extraction of estrogen by PDMS in the culture media.

In summary, there is a need to develop devices or substrates with micro-structured surfaces that possess PDMS or PDMS-like chemistry favorable for in-vivo like cell culture and that are free of tendency to remove hydrophobic molecules from the aqueous environment so that cell culture articles having such surfaces can be used in a broad applications including cytotoxicity studies of hydrophobic pharmaceutical compounds.

### BRIEF SUMMARY

Among other things, the present disclosure describes cell culture articles having a micro-structured hydrophobic elastomeric surface, such as a micro-structured PDMS surface, that promotes three dimensional cell growth and that does not extract a large amount of hydrophobic molecules from aqueous media in contact with the surface. The micro-structured cell culture surface is a thin coating of hydrophobic elastomer, which is sufficiently thin so that the coating does not absorb or extract a large amount of hydrophobic molecules. The coating may be disposed on a non-absorbing or non-extracting micro-structured substrate.

In embodiments described herein, a cell culture article includes a substrate having a micro-structured surface and a coating disposed on the micro-structured surface of the substrate. The coating forms a micro-structured cell culture surface. The coating comprises PDMS and has an average thickness of between 5 micrometers and 100 micrometers.

In embodiments described herein, a cell culture article includes a substrate having a micro-structured surface and a hydrophobic elastomeric coating disposed on the micro-structured surface of the substrate. The coating forms a micro-structured cell culture surface. The coating has a water contact angle of greater than 20°, a thickness of between 5 micrometers and 100 micrometers, and an ultimate tensile modulus of less than 1 gigapascals.

In embodiments described herein, a method for manufacturing a cell culture article includes providing a substrate having a micro-structured surface, and coating the micro-structured surface of the substrate with a thin layer of a polymer comprising polydimethylsiloxane to produce a micro-structured cell culture surface. The layer is sufficiently thin such that the coating absorbs less than 25% of nefazodone in a phosphate buffered saline solution at a concentration of 200 micromolar after 24 hours of incubation on the cell culture surface.

In embodiments described herein, a method for manufacturing a cell culture article includes providing a substrate having a micro-structured surface and coating the micro-structured surface of the substrate with a thin layer of a polymer to produce a micro-structured cell culture surface. The polymer has a water contact angle of greater than 20°. The layer is sufficiently thin such that the coating absorbs less than 25% of nefazodone in a phosphate buffered saline solution at a concentration of 200 micromolar after 24 hours of incubation on the cell culture surface.

One or more embodiments of the cell culture articles, compositions, or methods described herein provide one or more advantages over prior cell culture articles, compositions, methods for producing cell culture articles, methods for culturing cells, or the like. For example, the cell culture surfaces described herein, in various embodiments, provide an environment to promote in vivo-like cell culture, in which cells may exhibit in vivo-like morphology not observed on traditional two dimensional cell culture substrates. The surfaces described herein may also, in embodiments, impose minimal disruption to hydrophobic components of surrounding aqueous environments, allowing the surfaces to be meaningfully used in a variety of applications such as cytotoxic studies of pharmaceutical compounds. These and other advantages will be readily understood from the following detailed description when read in conjunction with the accompanying drawings.

### BRIEF DESCRIPTION OF THE DRAWINGS

FIGS. 1-2 are schematic drawings of cross sectional views of coated cell culture articles in accordance with embodiments described herein.

FIG. 3A is a a top-down image of a silicon master used to generate a micro-structured substrate as discussed in the Examples.

FIG. 3B is a schematic cross sectional view of the silicone master shown in FIG. 3A.

FIGS. 4A-C are schematic sectional diagrams illustrating an embodiment of a process for coating a substrate.

FIG. 5 is a bar graph showing the amount of nefazodone remaining in phosphate buffered saline after 24 hours of incubation with various cell culture surfaces.

FIG. 6 is a bar graph showing the amount of nefazodone remaining in phosphate buffered saline after 24 hours of incubation with PDMS cell culture surfaces having varying thicknesses.

FIG. 7 is a plot showing the amount of nefazodone remaining in phosphate buffered saline after 24 hours of incubation with cell culture surfaces having varying water contact angles.

FIG. 8 is a plot showing the amount of nefazodone remaining in phosphate buffered saline after 24 hours of incubation with cell culture surfaces having varying ultimate tensile modulus.

FIG. 9A is an image of MCF-10A cells cultured on a micro-structured PDMS coated polystyrene surface.

FIG. 9B is an image of MCF-10A cells cultured on an uncoated micro-structured polystyrene surface.

FIG. 10A is an image of MCF-10A cells cultured on a flat polystyrene surface.

FIG. 10B is an image of MCF-10A cells cultured on flat PDMS surface.

FIGS. 11A-D are images of MCF-10A cells cultured on micro-structured PDMS coated polystyrene surfaces having varying PDMS thicknesses: no PDMS (11A); about 5 micrometer thick PDMS coating (11B); about 20 micrometer thick PDMS coating (11C); and about 50 micrometer thick PDMS coating (11D).

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

1. Definitions

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like. As used herein, “consisting essentially of,” as it relates to a composition, coating, article, method, or the like, means that the components of the composition, coating, article, method, or the like are limited to the enumerated components and any other components that do not materially affect the basic and novel characteristic(s) of the composition, coating, article, method, or the like. By way of example, items that may materially affect the basic properties of the components of a cell culture article, substrate or coating described herein are those components that may impart undesirable characteristics to such an article or substrate. For example, if the article, coating or substrate is clearly intended to promote in-vivo-like cell growth or to minimize extraction of hydrophobic molecules out of an aqueous environment, a component that results reduction of in-vivo-like characteristics of the cells or that increases the amount of hydrophobic molecules extracted from the aqueous environment may be considered to materially affect the basic and novel properties of the article, coating or substrate.

It will be understood that an article, substrate or coating that “consists of,” for example, a polymer may contain components other than polymerized monomers, as small or trace amounts of solvents, initiators, or the like may remain in the polymer after it is formed. Accordingly, an article, substrate or coating that “consists of” a polymer may contain the polymer and components that are incidental to forming the polymer.

Specific and preferred values disclosed for components, ingredients, cell types, properties, and like aspects, and ranges thereof, are for illustration only; they do not exclude other defined values or other values within defined ranges. The compositions, apparatuses, systems and methods of the disclosure include those having any value or any combination of the values, specific values, more specific values, and preferred values described herein.

As used herein, a “micro-structured surface” is a surface that has patterned topographical micro-features on the surface. “Micro-features” are features having dimensions less than 1 millimeter, such as less than 500 micrometers. In embodiments, the micro-features are less than 250 micrometers. The micro-features may be cavities having an inner diametric dimension of less than one millimeter. The micro-features may be troughs, projections, or the like.

As used herein, a “hydrophobic elastomer” is an elastomer that forms a surface having a water contact angle of greater than 20°, such as greater than 60°. An “elastomer” is a polymer having elastic properties similar to natural rubber, as generally understood in the art. In embodiments, elastomers have an ultimate tensile modulus of, for examples, less than 1 gigapascal, less than 500 megapascals, less than 100 megapascals, less than 50 megapascals, less than 20 megapascals, less than 10 megapascals or less than 5 megapascals.

As used in the claims presented herein below, “providing” a component means to make, purchase, or otherwise obtain the component.

Any direction referred to herein, such as “top,” “bottom,” “left,” “right,” “upper,” “lower.” And other directions and orientations are described herein for clarity in reference to the figures and are not to be limiting of an actual device or system or use of the device or system. Devices and systems as described herein may be used in a number of directions and orientations.

The use of headers herein is not intended to be limiting. For example, relevant discussion of a property, characteristic, component or the like of a coating may be provided under the heading “cell culture” rather than under the heading of “coating”. One or more embodiments of coatings described herein may include such a property, characteristic, component or the like, even though such discussion is not provided under the heading “coating”.

2. Cell Culture Article

The present disclosure describes, among other things, cell culture articles having a micro-structured hydrophobic elastomeric surface, such as a micro-structured PDMS surface, that promotes three dimensional cell growth and that does not extract a large amount of hydrophobic molecules from aqueous media in contact with the surface. The micro-structured cell culture surface is a thin coating of hydrophobic elastomer, which is sufficiently thin so that the coating does not absorb or extract a large amount of hydrophobic molecules. The coating may be disposed on a non-absorbing or non-extracting micro-structured substrate.

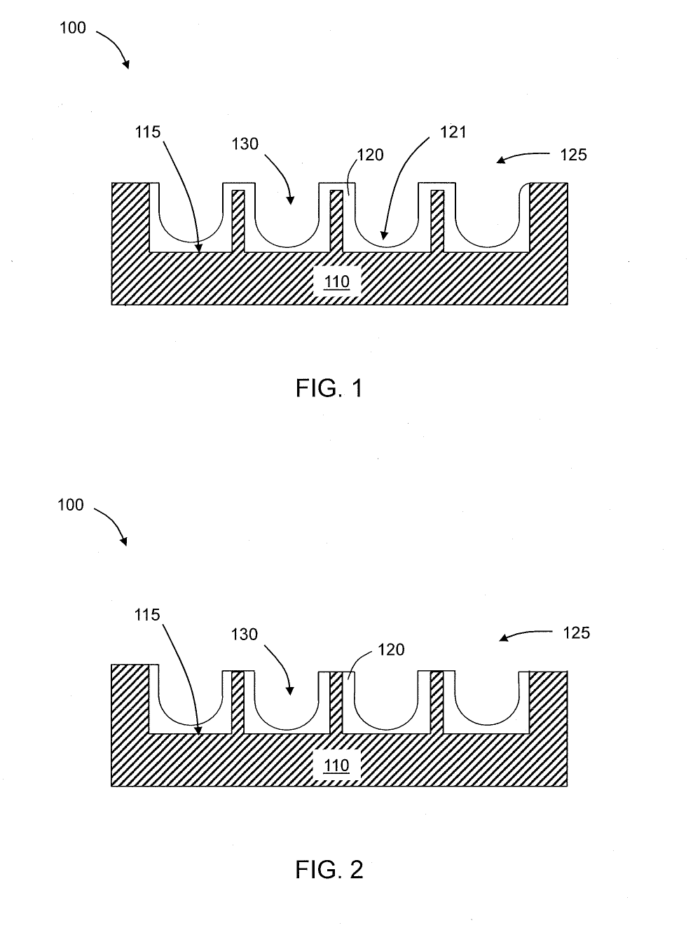

For example and with reference to FIGS. 1-2, schematic cross-sectional views of cell culture apparatuses 100 are shown. The cell culture apparatuses 100 include a substrate 110 having a micro-structured surface 115. A hydrophobic elastomeric coating 120 is disposed on the substrate 110 and forms a micro-structured cell culture surface 125 that assumes the general configuration of the micro-structured surface 115 of the substrate 110.

In the embodiment depicted in FIG. 1, the coating 120 is continuous and forms a single layer over a plurality of the depicted micro-features (micro-cavities or micro-wells are depicted). In the embodiment depicted in FIG. 2, the coating 120 is discontinuous and coats the inner surfaces of the depicted micro-cavities or micro-wells. While the overall coating 120 in FIG. 2 is discontinuous, the coating within each micro-cavity is preferably continuous. In either case, the coating (whether continuous or discontinuous) will be considered to form a micro-structured cell culture surface 125.

As shown, in e.g. FIG. 1, the bottom portion 121 of the coating 120 in the micro-well or micro-cavity may be concave despite the flat or rectangular shape of the bottom portion of the underlying substrate 110 in the micro-cavity. The concave shape may, in some cases, result from a meniscus effect of the coating process employed. Such a concave shape may be desirable in embodiments to encourage or facilitate cell-cell interaction and cell clustering.

Any suitable cell culture article may include a substrate 110 having a micro-structured surface and a hydrophobic elastomeric coating 120 is disposed on the substrate 110 that forms a micro-structured cell culture surface 125 that assumes the general configuration of the micro-structured surface 115 of the substrate 110. Examples of such suitable cell culture articles include single and multi-well plates, such as 6, 12, 96, 384 and 1536 well plates, jars, Petri dishes, beakers, roller bottles, slides, such as chambered and multi-chambered culture slides, tubes, cover slips, membranes, microcarriers, cups, spinner bottles perfusion chambers, bioreactors, CellSTACK® and fermenters. In embodiments, the structure of the cell culture article 100 is formed from the material forming the substrate 110 and is contiguous with the substrate. In embodiments, the structure of the culture article 100 is formed from a part separate from the substrate 110 and the substrate is placed in, attached to, adhered to, or otherwise affixed to the structural portion of the article. In such cases, the coating 120 may be applied before or after associating the substrate 110 with the structural portion of the article.

3. Coating

Any suitable hydrophobic elastomeric coating may be employed in accordance with the teachings presented herein. As described in more detail below in the Examples, forming a thin coating of hydrophobic elastomer on a micro-structured substrate may provide a surface suitable for promoting or facilitating three dimensional cell growth, while extracting lower amounts of hydrophobic molecules from aqueous medium than counterpart cell culture surfaces fabricated entirely from the hydrophobic elastomeric polymer (without the micro-structured substrate). While the Examples below employ a polydimethylsiloxane (PDMS) coating, it is believed that similar benefits may be obtained with other hydrophobic elastomeric coatings.

It is believed that PDMS serves as a suitable substrate for promoting in vivo-like cell morphology or functionality because of its surface and other properties. While not intended to be bound by theory, it is believed that cells do not attach, or do not strongly attach, to PDMS because of its hydrophobic properties and it is believed that such a surface encourages cell-cell interaction rather than attachment to the PDMS surface. In addition, PDMS is “soft” and may provide a more in vivo-like environment relative to harder materials that are often used for cell culture. Further, PDMS is gas (oxygen and carbon dioxide) permeable and may facilitate exchange of gases to and from the cells to facilitate cell growth in culture. Other hydrophobic elastomeric polymers having one or more properties similar to PDMS may be beneficially used for purposes of coating a micro-structured substrate.

As discussed in more detail below in the EXAMPLES, it has been found that coating thickness, modulus and hydrophobicity may affect the amount of hydrophobic molecules the coating absorbs or extracts from an aqueous medium in contact with the coating. Thickness, modulus and hydrophobicity also appear to be relevant variables for cell culture. Thus, a balance between an acceptable amount of absorption of hydrophobic molecules and desired cell culture properties may be weighted in determining a suitable thickness, modulus and hydrophobicity of a coating.

In embodiments, a suitable elastomeric polymer may be hydrophobic. For example, the elastomeric polymer may form a surface having a water contact angle of 20° or more, 30° or more, 40° or more, 50° or more, 60° or more, 70° or more or 80° or more. For example, the hydrophobic elastomer may form a surface having a water contact angle of between about 20° and about 130°, between about 60° and about 130°, or between about 80° and 130°.

In embodiments, a suitable elastomeric polymer is “soft”. For example, the polymer may have an ultimate tensile modulus of 1 gigapascals or less, 500 megapascals or less, or about 30 pascals. It will be understood that, in embodiments, the amount of cross-linking may be varied to adjust the modulus of the resulting polymer to achieve a desirable soft polymer.

Examples of hydrophobic elastomers that may be employed include polymers or copolymers of: PDMS; a polyurethane; a poly(tetrafuoroethylene); a poly(methyl methacrylate); a silicone rubber; a polyethylene glycol; a polyacrylic; a poly(vinyl chloride); a polyethylene; and a polypropylene.

Regardless of the polymer used to form the coating, the polymer may be formed in-situ on the surface of the substrate (e.g., by polymerizing one or more monomer, oligomer, or prepolymer), may be formed by disposing a dissolved polymer on the surface of the substrate, or the like.

Preferably, the coating is sufficiently thin to avoid a large amount of hydrophobic small molecules from being extracted from an aqueous medium by the coating (e.g., via adsorption or absorption). In embodiments, 70% or more, 75% or more, 80% or more, 85% or more, 90% or more or 95% or more of a hydrophobic small molecule (such as a pharmaceutical agent) remains in the aqueous medium after 24 hours of contact with the coating. Examples of hydrophobic small molecules include estrogen, paclitaxel, doxorubicin, and nefazadone. In embodiments, the coating is sufficiently thin that the coating absorbs less than 30% of nefazodone, 1-(3-[4-(3-chlorophenyl)piperazin-1-yl]propyl)-3-ethyl-4-(2-phenoxyethyl)-1H-1,2,4-triazol-5(4H)-one, in a phosphate buffered saline solution at a concentration of 200 micromolar after 24 hours of incubation on the cell culture surface. That is, 70% or more remains in the aqueous medium.

While the thickness of the coating required to achieve a desired limited amount of absorption or extraction of hydrophobic molecules may vary depending on the composition of the coating, the thickness of the coating should not be so great such that the coating effectively masks the microstructure of the underlying substrate, rendering the coating surface deficient in microstructure for desired cell culture purposes. In embodiments, the average thickness of the coating is less than about 150 micrometers, less than about 125 micrometers, less than about 100 micrometers, less than about 90 micrometers, less than about 80 micrometers, less than about 70 micrometers, less than about 60 micrometers, less than about 50 micrometers, less than about 40 micrometers, less than about 30 micrometers, or less than about 20 micrometers.

The coating is also preferably thick enough to form a suitable surface for cell culture. In various embodiments, the coating has an average thickness of about 2 micrometers or greater, about 3 micrometers or greater, about 4 micrometers or greater, about 5 micrometers or greater, about 6 micrometers or greater, about 7 micrometers or greater, about 8 micrometers or greater, about 9 micrometers or greater, or about 10 micrometers or greater.

In embodiments, the average thickness of the coating is from about 2 micrometers to about 150 micrometers.

The thickness of the coating may be controlled in any suitable manner. It will be understood that the process employed; e.g., spraying, dipping, casting, etc., may affect the thickness of the coating or the ability to control the thickness of the coating. It will be further understood that the amount or concentration of polymer, prepolymer, oligomer, or monomer used may affect the thickness of the coating.

In embodiments, a suitable hydrophobic elastomeric coating is gas permeable. For example, coating may have an oxygen permeability of 0.5×10⁹, cm³ cm/(s cm² cm Hg) or greater at 25° C., 1 atmosphere, such as between about 0.5×10⁹, cm³ cm/(s cm² cm Hg) to about 200×10⁹, cm³ cm/(s cm² cm Hg)). In embodiments, the coatings may be porous or rendered porous to increase gas permeability or otherwise enhance cell culture properties. A polymer may be made porous via any suitable mechanism, such as mixing with gas; foaming; use of a pore-forming agent which is later extracted, dissolved, or the like; or the like, prior to, during or after polymerization.

In embodiments, the micro-structured culture surface of the coating is treated or coated to impart a desirable property or characteristic to the treated or coated surfaces. Examples of surface treatments often employed for purposes of cell culture include corona or plasma treatment. In embodiments, the micro-structured culture surface of the coating are coated with extracellular matrix (ECM) materials, such as naturally occurring ECM proteins or synthetic ECM materials. The type of EMC selected may vary depending on the desired result and the type of cell being cultures, such as a desired phenotype of the culture cells. Examples of naturally occurring ECM proteins include fibronectins, collagens, proteoglycans, and glycosaminoglycans. Examples of synthetic materials for fabricating synthetic ECMs include polyesters of naturally occurring α-hydroxy acids, poly(DL-lactic acid), polyglycolic acid (PGA), poly-lactic acid) (PLLA) and copolymers of poly(lactic-co-glycolic acid) (PLGA). Such thermoplastic polymers can be readily formed into desired shapes by various techniques including molding, extrusion and solvent casting. Amino-acid-based polymers may also be employed in the fabrication of an ECM for coating a projection or substrate. For example, collagen-like, silk-like and elastin-like proteins may be included in an ECM. In various embodiments, an ECM includes alginate, which is a family of copolymers of mannuronate and guluronat that form gels in the presence of divalent ions such as Ca²⁺. Any suitable processing technique may be employed to fabricate ECMs from synthetic polymers. By way of example, a biodegradable polymer may be processed into a fiber, a porous sponge or a tubular structure.

One or more ECM material may be used to coat the micro-structured culture surface of the coating. Cell adhesion factors, such as polypeptides capable of binding integrin receptors including RGD-containing polypeptides, or growth factors can be incorporated into ECM materials to stimulate adhesion or specific functions of cells using approaches including adsorption or covalent bonding at the surface or covalent bonding throughout the bulk of the materials.

In embodiments, the micro-structured culture surface of the coating is not treated or is not coated.

4. Substrate

Any suitable substrate having a micro-structured surface may be employed in accordance with the teachings presented herein. The substrate may be formed from any suitable material, including metal, glass, ceramic or polymeric material. Preferably the material is compatible with cells, cell culture media and agent that may be employed in cell culture or assays involving cultured cells.

Examples of suitable polymeric materials for cell culture include polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, and copolymers thereof, nitro celluloses, polymers of acrylic and methacrylic esters, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methylmethacrylate), poly(ethylmethacrylate), poly(butylmethacrylate), poly(isobutylmethacrylate), poly(hexlmethacrylate), poly(isodecylmethacrylate), poly(laurylmethacrylate), poly(phenylmethacrylate), poly(methacrylate), poly(isopropacrylate), poly(isobutacrylate), poly(octadecacrylate), polyethylene, polypropylene poly(ethylene glycol), poly(ethylene oxide), poly(ethylene terephthalate), poly(vinyl alcohols), poly(vinyl acetate), poly vinyl chloride, polystyrene, polyhyaluronic acids, casein, gelatin, gluten, polyanhydrides, polyacrylic acid, alginate, chitosan, and any copolymers thereof, or any combination thereof.

In embodiments, the substrate is stiffer than the coating to provide structural support for the thin coating. By way of example, the support may have an ultimate tensile modulus of 1 gigapascals or more.

In some embodiments, the substrate is gas permeable. Examples of gas permeable polymers that may be used to form the substrate include polytetrafluoroethylene (PTFE) and polymethylpentene (PMP). Of course other polymers may be made porous to increase gas permeability (e.g., as described above under the heading “coating”).

The surface of the substrate may be micro-structured employing any suitable technique. For example, micro-features may be imparted on or to the surface of the substrate via molding, embossing, casting and curing, or the like. To form suitable molds having micro-scale features, a master, such as a silicon master, may be formed by proximity IV photolithography. By way of example, a thin layer of photoresist, an organic polymer sensitive to ultraviolet light, may be spun onto a silicon wafer using a spin coater. The photoresist thickness is determined by the speed and duration of the spin coating. After soft baking the wafer to remove some solvent, the photoresist may be exposed to ultraviolet light through a photomask. The mask's function is to allow light to pass in certain areas and to impede it in others, thereby transferring the pattern of the photomask onto the underlying resist. The soluble photoresist is then washed off using a developer, leaving behind a protective pattern of cross-linked resist on the silicon. At this point, the resist is typically kept on the wafer to be used as the topographic template for molding the stamp. Alternatively, the unprotected silicon regions can be etched, and the photoresist stripped, leaving behind a wafer with patterned silicon making for a more stable template. The lower limit of the features on the structured substrates is dictated by the resolution of the fabrication process used to create the template. This resolution is determined by the diffraction of light at the edge of the opaque areas of the mask and the thickness of the photoresist. Smaller features can be achieved with extremely short wavelength UV light (-200 nm). For submicronic patterns (e.g. etch depths of about 100 nanometers), electron beam lithography on PMMA (polymethylmetacrylate) may be used. Templates can also be produced by micromachining, or they can be prefabricated by, e.g., diffraction gratings.

To enable simple demoulding of the master, an anti-adhesive treatment may be carried out using silanisation in liquid phase with OTS (octadecyltrichlorosilane) or fluorinated silane, for example. After developing, the wafers may be vapor primed with fluorinated silane to assist in the subsequent removal of the array of projections. Examples of fluorinated silane that may be used include, but are not limited to, (tridecafluoro-1,1,2,2-tetrahydroctyl)trimethoxysilane, and tridecafluoro-1,1,2,2-tetrahydrooctyl)triethoxysilane.

The micro-structured substrate may be molded, stamped, embossed, or the like from the master or from a negative replica of the master. A negative replica of the master may be created from any suitable material such as an inorganic material or polymeric material.

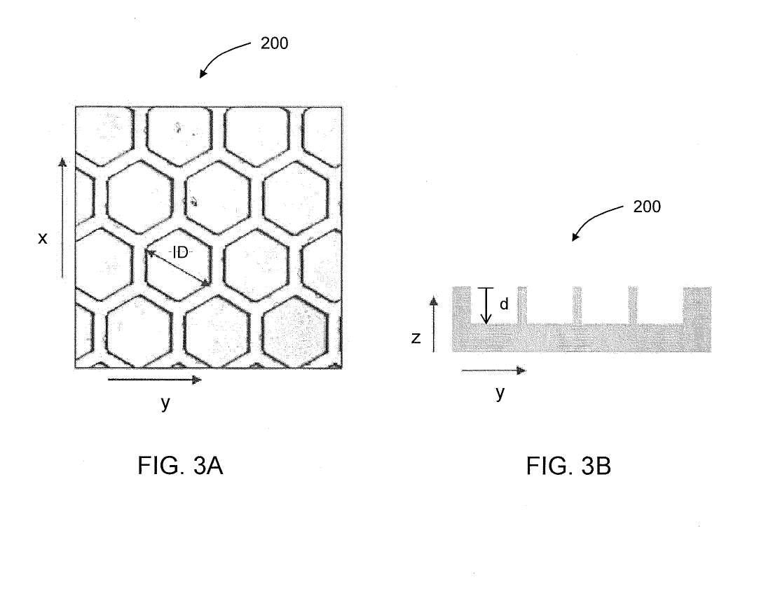

By way of example and with reference to FIGS. 3A-B, a top-down image of a silicon master 200 (3A) and a schematic cross sectional view of the silicon master 200 (FIG. 3B) are shown. The silicon master 200 depicted in FIG. 3A was used in the Examples below to create a micro-structured polystyrene substrate. The substrate was generated via hot embossing using a PDMS negative replica of the silicon master 200. In the depicted embodiment, the silicon master 200 (and thus the resulting substrate) has micro-cavities having an inner diametric dimension (ID) of about 150 micrometers and a depth (d) of about 150 micrometers. The depicted micro-cavities are honeycomb shaped or hexagonal, but may be any other shape such as circular, square, octagonal, etc.

The micro-features of a structured surface of the substrate may be of any suitable size or shape. It will be understood that the size and shape of the micro-features of a micro-structured surface may vary depending on the cells cultured. For example, microcavities having an inner diametric dimension of less than about 30 micrometers may not support three dimensional culture of breast cells, but may be suitable for supporting three dimensional culture of hepatocytes. Diametric dimensions greater than 100 micrometers may more favorably result in in-vivo-like morphology of breast cells.

The dimensions of the micro-features of the structured surface of the substrate should, in some cases, take into account the thickness of the coating that is to be applied. For example, if the micro-features of the substrate are cavities and if the thickness of the coating is, for example, 25 micrometers, then the resulting structured surface of the coating will, in some cases, have microcavities having inner diametric dimensions of about 30 micrometers less than that of the substrate. Accordingly, the micro-feature dimensions of the coating may differ from the micro-feature dimensions of the substrate. The micro-feature dimensions of the substrate may readily be adjusted to account for the desired micro-feature dimensions of the coating.

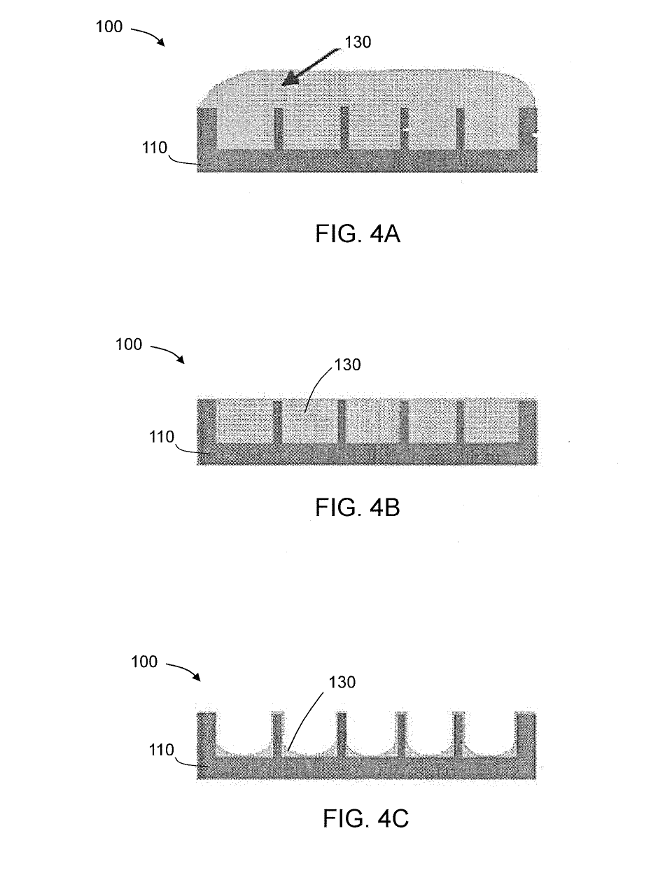

The coating may be applied to the substrate via any suitable process, such as casting, spraying, embossing, dipping, or the like. By way of example and with reference to FIGS. 4A-C, a process used to generate the coated substrate used in the Examples below is depicted. In the depicted method, PDMS prepolymer solution 130 (e.g. 10% in hexane) was dispensed on the substrate 110 (4A), excess pre-polymer 130 was removed (4B), and the solvent was evaporate and the PDMS 120 cured. The resulting article 100 was used to culture breast cells as discussed in the Examples below.

Of course it will be understood that the method depicted in FIGS. 4A-C is only one suitable method for coating a substrate and that other suitable methods may be readily employed.

5. Cell Culture

A cell culture article as described above may be seeded with cells. The cells may be of any cell type. For example, the cells may be connective tissue cells, epithelial cells, endothelial cells, hepatocytes, skeletal or smooth muscle cells, heart muscle cells, intestinal cells, kidney cells, or cells from other organs, stem cells, islet cells, blood vessel cells, lymphocytes, cancer cells, or the like. The cells may be mammalian cells, preferably human cells, but may also be non-mammalian cells such as bacterial, yeast, or plant cells. In numerous embodiments, the cells are mammary epithelial cells. As used herein, mammary epithelial cells include primary cells, immortalized cell lines, and breast cancer cells having an epithelial origin. Breast cancer cells can be invasive or non-invasive.

Prior to seeding cells, the cells may be harvested and suspended in a suitable medium, such as a growth medium in which the cells are to be cultured once seeded onto the surface. For example, the cells may be suspended in and cultured in a serum-containing medium, a conditioned medium, or a chemically-defined medium. One or more growth or other factors may be added to the medium as desired.

The cells may be seeded at any suitable concentration. Typically, the cells are seeded at about 1,000 cells/cm² of substrate to about 500,000 cells/cm². For example, cells may be seeded at about 1,000 cells/cm² of substrate to about 150,000 cells/cm². However, higher and lower concentrations may readily be used. The incubation time and conditions, such as temperature, CO₂ and O₂ levels, growth medium, and the like, will depend on the nature of the cells being cultured and can be readily modified. The amount of time that the cells are incubated on the surface may vary depending on the cell response desired.

Embodiments of cell culture articles as described herein are capable of supporting culture of mammary epithelial cells, where such cells exhibit in-vivo-like morphology or characteristics, such as formation of acini structures in non-malignant mammary epithelial cells, formation of mass cell structures with robust cell-cell interaction and disorganized nuclei in non-invasive breast cancer cells, formation of elongated cell bodies resembling invasive processes in invasive malignant breast cancer cells, response to anti-cancer agents by breast cancer cells, or reversion of malignant phenotype of breast cancer cells. Acinus structures are clusters of cells that resemble a many-lobed berry, such as a raspberry. In-vivo, mammary epithelial cells that form acini form the tissue of the breast gland that produce fluid or milk.

The cultured cells may be used for any suitable purpose, including investigational studies of the effects of the cell culture surface on the cells, the effect of the cultured cells on known or potential therapeutic agents, and the effect of known or potential therapeutic agents on the cultured cells.

6. Overview of Aspects of Disclosure

In a first aspect, a cell culture article includes a substrate having a micro-structured surface and a coating disposed on the micro-structured surface of the substrate. The coating forms a micro-structured cell culture surface. The coating comprises PDMS and has an average thickness of between 5 micrometers and 100 micrometers.

A second aspect is a cell culture article of the first aspect, wherein the coating has an average thickness of between 10 micrometers and 50 micrometers.

A third aspect is a cell culture article of the first or second aspect, wherein the coating consists of polydimethylsiloxane.

In a fourth aspect, a cell culture article includes a substrate having a micro-structured surface and a hydrophobic elastomeric coating disposed on the micro-structured surface of the substrate. The coating forms a micro-structured cell culture surface. The coating has a water contact angle of greater than 20°, a thickness of between 5 micrometers and 100 micrometers, and an ultimate tensile modulus of less than 1 gigapascal, less than 500 megapascal, less than 100 megapascal, less than 50 megapascal, less than 20 megapascal or less than 10 megapascals.

A fifth aspect is a cell culture article of the fourth aspect, wherein water contact angle is greater than 30°.

A sixth aspect is a cell culture article of the fourth aspect, wherein water contact angle is between 60° and 130°.

A seventh aspect is a cell culture article of the fourth, fifth or sixth aspect, wherein the coating has an oxygen permeability of 0.5×10⁹, cm³ cm/(s cm² cm Hg) or greater at 25° C., 1 atmosphere.

A eighth aspect is a cell culture article of any of aspects 4-7, wherein the coating has an ultimate tensile modulus of less than 5 megapascals.

A ninth aspect is a cell culture article of any of aspects 4-8, wherein the coating absorbs less than 25% of nefazodone in a phosphate buffered saline solution at a concentration of 200 micromolar after 24 hours of incubation on the cell culture surface.

A tenth aspect is a cell culture article of any of aspects 4-9, wherein the substrate comprises polystyrene.

A eleventh aspect is a cell culture article of any of aspects 4-10, wherein the article is free of components of unknown origin.

In a twelfth aspect, a method includes seeding cells on the cell culture surface of the article of any of aspects 1-11, and contacting the seeded cells with cell culture medium.

A thirteenth aspect is a method of the twelfth aspect, wherein the cell culture surface is configured to cause the cells to exhibit an in-vivo-like morphology.

A fourteenth aspect is a method of the twelfth aspect, wherein the cells are breast cells and wherein the cells form acinus structures when cultured on the cell culture surface.

In a fifteenth aspect, a method for manufacturing a cell culture article includes providing a substrate having a micro-structured surface, and coating the micro-structured surface of the substrate with a thin layer of a polymer comprising polydimethylsiloxane to produce a micro-structured cell culture surface. The layer is sufficiently thin such that the coating absorbs less than 25% of nefazodone in a phosphate buffered saline solution at a concentration of 200 micromolar after 24 hours of incubation on the cell culture surface.

A sixteenth aspect is a method of the fifteenth aspect, wherein the polymer consists of polydimethylsiloxane.

In a seventeenth aspect, a method for manufacturing a cell culture article includes providing a substrate having a micro-structured surface and coating the micro-structured surface of the substrate with a thin layer of a polymer to produce a micro-structured cell culture surface. The polymer has a water contact angle of greater than 20°. The layer is sufficiently thin such that the coating absorbs less than 25% of nefazodone in a phosphate buffered saline solution at a concentration of 200 micromolar after 24 hours of incubation on the cell culture surface.

An eighteenth aspect is a method of the seventeenth aspect, wherein the polymer layer has an ultimate tensile modulus of less than 5 megapascals.

A nineteenth aspect is a method of the seventeenth or eighteenth aspect, wherein the polymer layer has an average thickness of between 5 micrometers and 100 micrometers.

A twentieth aspect is a method of any of aspects 17-19, wherein providing the substrate having the micro-structured surface comprises generating the substrate via molding.

In the following, non-limiting examples are presented, which describe various embodiments of the compositions, articles, and methods discussed above.

### EXAMPLES

1. Breast Cell MCF-10A Culture Medium

MCF-10A cells were cultured in culture medium DMEM/F12 (Invitrogen #11965-118) with 5% horse serum (Invitrogen #16050-122). 5 milliliters of Pen/Strep (100× solution, Invitrogen #15070-063), 20 nanogram/milliliter of EGF, 0.5 microgram/milliliter of hydrocortisone, 100 nanogram/milliliter of cholera toxin and 10 microgram/milliliter of insulin,

2. Fabrication of Polystyrene Substrate with Arrays of Microcavities with a Thin Layer of PDMS Coating

A silicon master mold shown in FIG. 3A was generated via a micro-fabrication process. The master includes micro-cavities having outer diametric dimensions (OD) of about 150 microns and depths of about 150 microns.

A PDMS negative replica of the silicon mold was used to generate array of micro cavities in a polystyrene (PS) sheet using a hot embossing process. The PS substrate with micro cavities was cut to proper size using a puncher and attached to the bottom of a 96-well tissue culture (TCT) plate by double adhesive tape.

A commercial Sylgard 184 kit was used to prepare PDMS pre-polymer mixtures of 2%, 7.5%, 15% and 30% (by weight) PDMS in hexane. Ten microliters of the PDMS pre-polymer solution was dispensed into each well (FIG. 4A), excess amount was removed (FIG. 4B), and remaining hexane solvent in the well was allowed to evaporate in a hood (FIG. 4C). Finally, the plate was cured in oven at 60° C. for 4 hours.

Nefazodone is a hydrophobic compound readily removed from aqueous buffer by PDMS in a matter of hours. Incubated with PDMS for 24 hours, typically less than 20% of the Nefazodone will remain in the buffer. To demonstrate the advantage of disclosed devices having minimal impact on Nefazodone concentration in buffer, 200 microliters of 200 micromolar Nefazodone-PBS solution was incubated for 24 hours in (i) PDMS coated PS micro-cavity wells, coated with 2% PDMS, 7.5% PDMS, 15% PDMS, and 30% PDMS; (ii) uncoated PS micro-cavity wells; (iii) uncoated TCT wells; iv) uncoated polystyrene wells; and (v) micro-cavity wells formed from PDMS bottom. UV-VIS adsorption at 260 nm was tested for each solution to measure the concentration of Nefazodone that remained in the solutions, or the retention of Nefazodone.

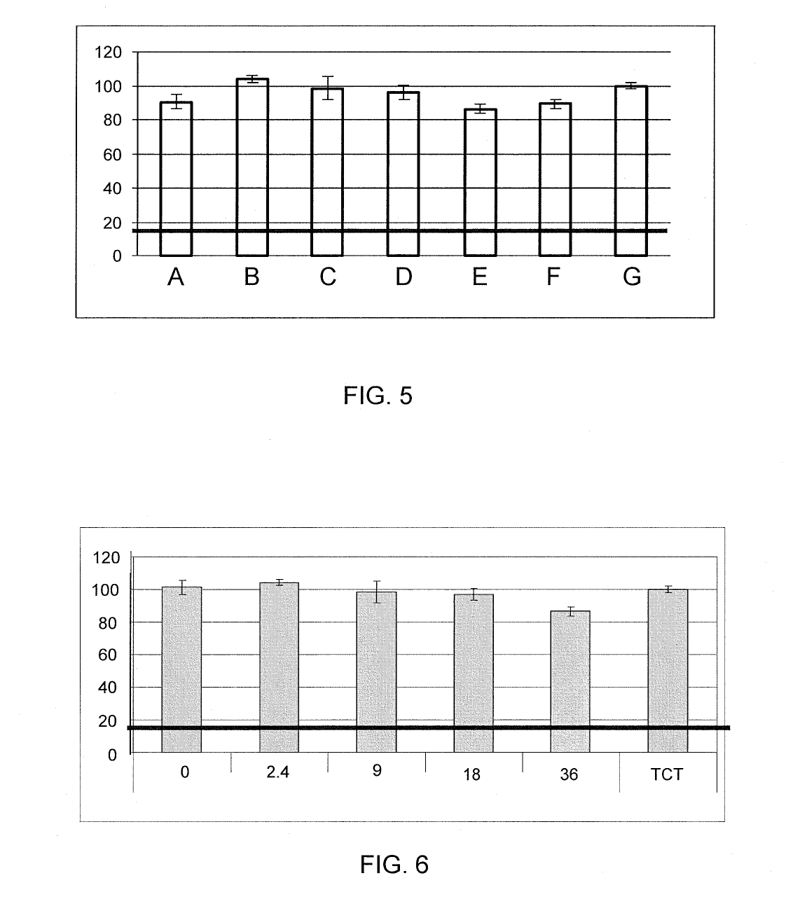

As shown in FIG. 5, in which the Y-axis represents the percentage of nefazodone remaining, less than 20% of the nefazodone remained in the aqueous buffer when incubated in the wells formed solely from PDMS (see thick horizontal bar). In contrast about 90% of the nefazodone remained in the aqueous buffer When incubated in the wells formed solely from polystyrene (A). Concentrations of Nefazodone remaining in aqueous buffer similar to polystyrene alone (A) were observed with polystyrene coated with a thin layer of PDMS (B: 2% PDMS; C: 7.5% PDMS; D: 15% PDMS; E: 30% PDMS). It is believed that higher weight percentages of PDMS in the coating solution results in thicker coatings. However, the results shown in FIG. 5, indicate that, even with a high weight percentage PDMS coating, much more Nefazodone remains in aqueous buffer (is not adsorbed) than when the microwells are formed from PDMS alone. A composite substrate with a thin layer of PDMS coating over non-absorbing material such as PS only causes negligible change of the hydrophobic compositions in the buffer, while still providing the desirable PDMS chemistry for cell culture, as discussed in more detail below.

In FIG. 5, F is a flat polystyrene holy plate, and G is a flat issue culture treated (TCT) polystyrene holy plate.

A similar study was performed where the thickness of the resulting PDMS coating was measured. The thicknesses were varied by varying the percentage of PDMS applied to the substrate. The results are presented in FIG. 6, where the percentage of nefazodone that remained in the aqueous buffer is shown in the X-axis and PDMS coating thickness (in micrometers) shown on the Y-axis. As with FIG. 5, the horizontal bar represents the amount of nefazodone that remained in the aqueous buffer when incubated in the wells formed solely from PDMS (less than 20%). The amount of nefazodone absorbed by the PDMS coating increased with increasing coating thickness. However, greater than 80% of nefazodone remained in the aqueous buffer when incubated in wells having coatings as thick as 36 micrometers.

3. Effect of Hydrophobicity on Retention of Hydrophobic Molecule

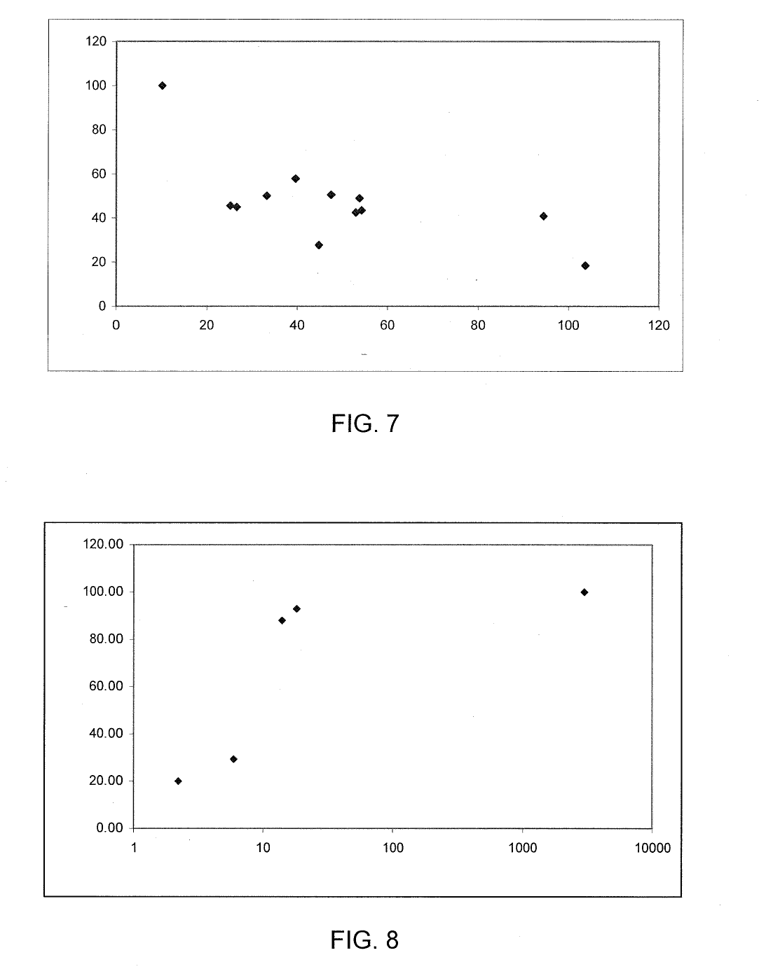

The effect of coating hydrophobicity on the retention of nefazodone was examined. Pieces of PDMS-PEG copolymers having a thickness of about 100 microns were incubated with nefazodone/PBS solutions as described above in Example 2. The PDMS-PEG copolymers contained varying ratios of PDMS and PEG with different cross-link percentages to produce coatings having a variety of water contact angles (depicted on X-axis in FIG. 7).

The PDMS/PEG copolymers produced are listed in the table below, where “single” crosslinker refers to tetraethoxysilane (TEOS), and “dual” crossliner refers to TEOS and bis[(3-methoxysilyl)propyl]polypropylene oxide (BMPPO), SIB1660.0.

| PDMS/PEG (mol/mol) | | | |
|---|---|---|---|
| PDMS | PEG | Crosslinker | Contact Angle |
| 1 | 0 | dual | 103.7 |
| 4 | 1 | dual | 47.5 |
| 2 | 1 | dual | 39.6 |
| 1 | 1 | dual | 33.2 |
| 1 | 2 | dual | 53.7 |
| 1 | 8 | dual | 44.8 |
| 1 | 0 | single | 94.5 |
| 4 | 1 | single | 52.9 |
| 2 | 1 | single | 54.2 |
| 1 | 1 | single | 26.6 |
| 1 | 2 | single | 25.2 |

As shown in FIG. 7, resulting polymers having a water contact angle of greater than about 20° resulted in substantial loss of nefazodone from the aqueous buffer (the percentage of nefazodone remaining is shown on the Y-axis). Accordingly, the composite cell culture articles described herein, where the article includes a thin coating serving as a cell culture surface, may produce beneficial effects (reduced absorption or extraction of hydrophobic molecules) for a variety of polymer coatings (those having contact angles of about 20° or greater).

4. Effect of Modulus on Retention of Hydrophobic Molecule

The effect of coating modulus on the retention of nefazodone was examined. In this study, a number of elastomers having similar water contact angle but various stiffness (ultimate tensile modulus) were incubated with nefazodone/PBS solutions as described above in Example 2. Elastomer pellets (all were pellets except for PDMS) of the same weight were directly incubated with nefazodone solution. The resulting polymers had water contact angles similar to PDMS (by observation with eyes).

The polymers used are presented in the table below:

| | Ultimate tensile | Retention of nefazodone |
|---|---|---|
| Polymer | strength (Mpa) | (%) |
| Versaflex ™ OM9-802CL | 6 | 29.51 |
| Vistamaxx ™ 400 | 18 | 92.94 |
| Vistamaxx ™ 6102 | 13.9 | 87.83 |
| PDMS | 2.24 | 20 |
| Polystyrene | 3000 | 100 |

As shown in FIG. 8, as the ultimate tensile modulus decreases (i.e. the coating polymer is “softer”), the amount of nefazodone lost from the aqueous buffer increases, which polymers having an ultimate tensile modulus of less than about 10 megapascal resulting in substantial loss of nefazodone from the aqueous buffer. In FIG. 8B, the Y-axis represents the percentage of nefazodone remaining, and the X-axis represents the ultimate tensile modulus in log (Mpa).

Accordingly, the composite cell culture articles described herein, where the article includes a thin coating serving as a cell culture surface, may produce beneficial effects (reduced absorption or extraction of hydrophobic molecules) for a variety of polymer coatings (those having an ultimate tensile modulus of about 10 megapascal or less).

5. Use of PDMS Coated PS Substrate for Culture and Formation of MCF-10 Acini

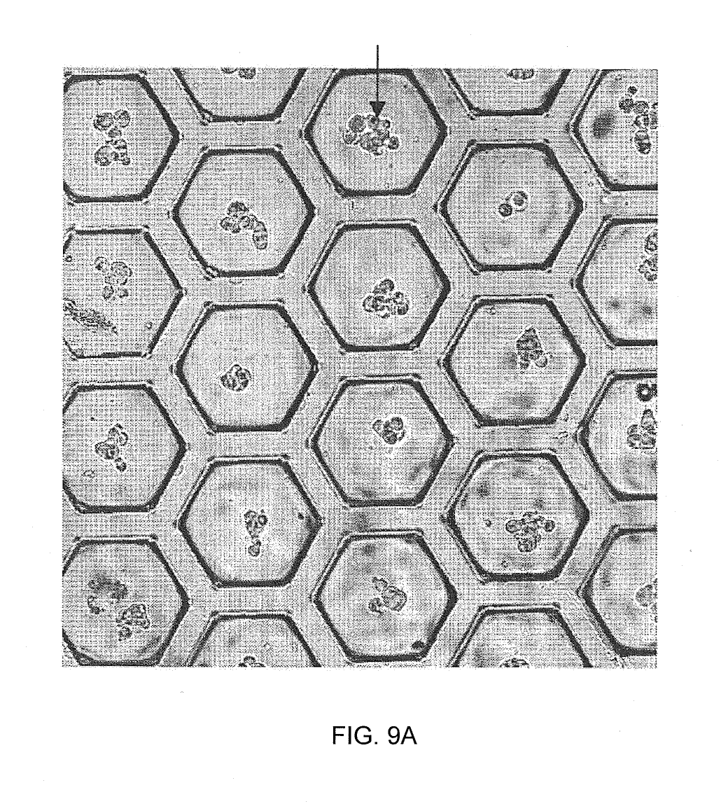

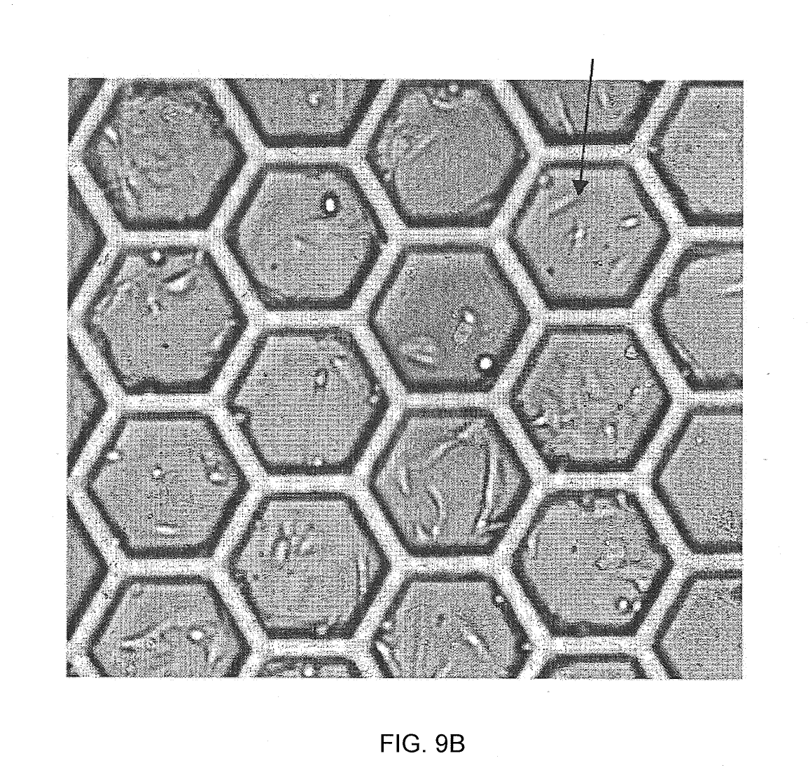

To demonstrate cell culture performance of the disclosed composite substrate with PDMS coating on PS, we cultured MCF-10A in PDMS coated PS micro-cavities as well as in uncoated PS micro cavities. The cell morphology on the two substrates was distinctively different as shown in FIGS. 9A-B. The cells spread and formed monolayer morphologyin PS microcavities. In contrast, the cells spread very little in the PDMS coated PS micro-cavities and formed cell aggregates similar to its in-vivo acini morphology.

Thus, not only does the composite substrate having a thin layer of thin layer of PDMS coating over non-absorbing material such as PS cause only negligible changes in the concentration of the hydrophobic components in the buffer, such composite substrates also provide the desirable PDMS chemistry for cell culture. Accordingly, the beneficial cell culture aspects of PDMS, which may include gas permeability, modulus, surface properties such as water contact angle, and the like, are maintained with such composite substrates, while undesirable aspects of adsorption and retention of hydrophobic compounds are reduced.

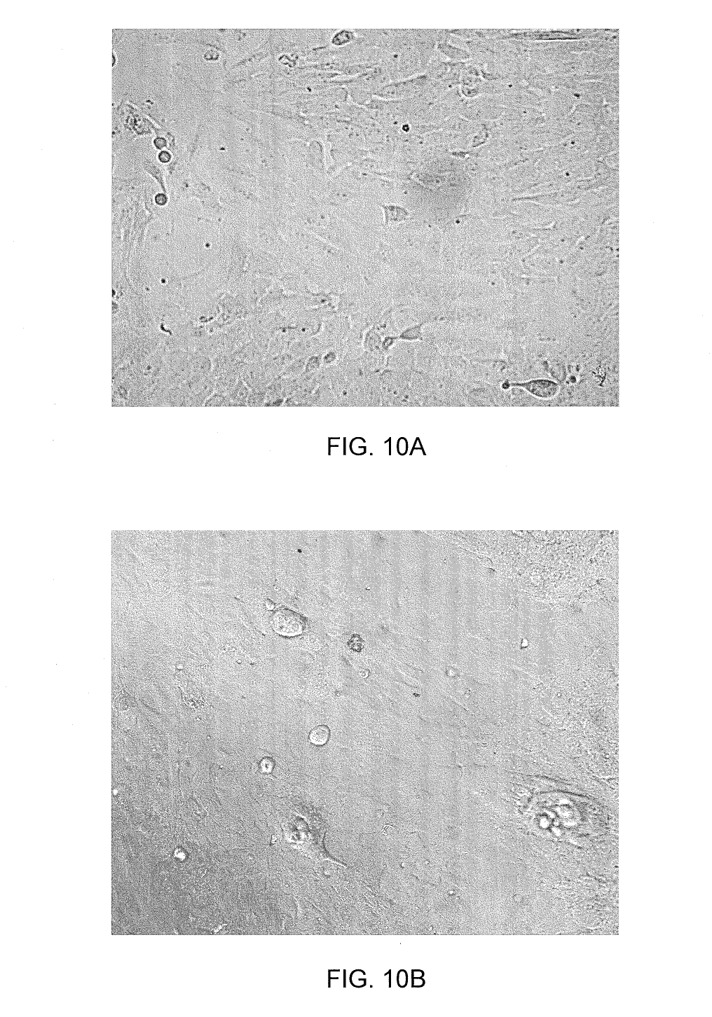

The benefit of a micro-structured surface was also determined, as morphology of cells cultured on flat PS (tissue culture treated—TCT) surfaces and fiat PDMS surfaces were compared to those cultured on micro-structured surfaces (see FIGS. 9A-B and associated text above). FIGS. 10A and 10B show results of cells cultured on a flat PS/TCT surface (10A) and flat PDMS (10B) surface. As shown in FIGS. 10A-B monolayers formed on both the flat PS/TCT and flat PDMS surfaces. Formation of acinis was only observed on the micro-structured surfaces having a hydrophobic elastomeric coating (see FIG. 9B and associated text above).

6. Effect of Coating Thickness on Culture and Formation of MCF-10 Acini

To evaluate cell culture performance of micro-structured hydrophobic elastomeric coatings, substrates were coated with varying thicknesses of PDMS, generally as described above. MCF-10A cells were cultured on the resulting articles (as described above with regard to EXAMPLE 5) and cell morphology was observed.

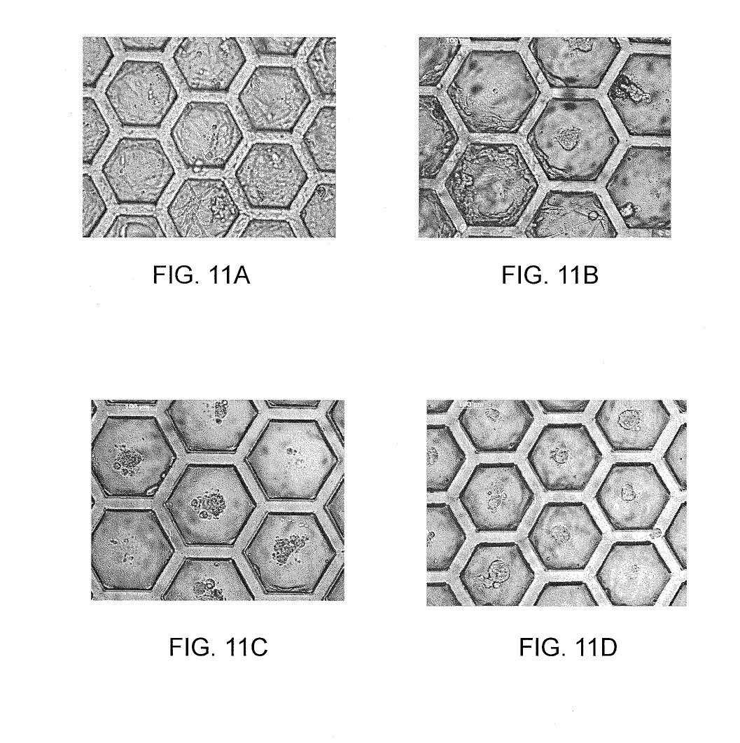

The results are presented in FIGS. 11A-D. FIG. 11A shows cells cultured on uncoated micro-structured polystyrene. FIG. 11B-D show cells cultured on PDMS coated micro-structured polystyrene. The average PDMS coating thickness of the article shown in FIG. 11B is about 5 micrometers The average PDMS coating thickness of the article shown in FIG. 11C is about 20 micrometers. The average PDMS coating thickness of the article shown in FIG, 11B is about 50 micrometers.

On the uncoated PS microcavities, the cells spread and formed a monolayer (FIG. 11A). On the articles having the PDMS coating of about 5 micrometers, acini were observed on some but not all microcavities (FIG. 11B). On the articles having a PDMS coating of greater than about 5 micrometers, acini were consistently observed on all the microcavities (FIGS. 11C-D).

Once a sufficient coating thickness is achieved, the cultured cells display in-vivo-like cell morphology. This may be due thicker coatings more uniformly coating the underlying substrate surface or due to a certain thickness being required to impart surface properties of the coating (as opposed to the substrate). Regardless of the reason, it appears that a minimum coating thickness is needed for in-vivo-like morphology to be consistently observed. In the case of PDMS, this minimum thickness is in the range of about 5 micrometers.

Thus, embodiments of COMPOSITE SUBSTRATE FOR 3D CELL CULTURE are disclosed. One skilled in the art will appreciate that the coatings, articles, compositions and methods described herein can be practiced with embodiments other than those disclosed. The disclosed embodiments are presented for purposes of illustration and not limitation.

What is claimed is:

1. A cell culture article comprising:
a substrate having a micro-structured surface; and
a coating disposed on the micro-structured surface of the substrate and forming a cell culture surface, wherein the cell culture surface is micro-structured,
wherein the coating comprises polydimethylsiloxane and has an average thickness of between 5 micrometers and 100 micrometers.

2. The cell culture article of claim 1, wherein the coating has an average thickness of between 10 micrometers and 50 micrometers.

3. The cell culture article of claim 1, wherein the coating consists of polydimethylsiloxane.

4. A cell culture article comprising:
a substrate having a micro-structured surface; and
a hydrophobic elastomeric coating disposed on the micro-structured surface of the substrate and forming a cell culture surface, wherein the cell culture surface is micro-structured,
wherein the coating has a water contact angle of greater than 20°, a thickness of between 5 micrometers and 100 micrometers, and an ultimate tensile modulus of less than 10 megapascals.

5. The cell culture article of claim 4, wherein water contact angle is greater than 30°.

6. The cell culture article of claim 4, wherein the water contact angle is between 60° and 130°.

7. The cell culture article of claim 4, wherein the coating has an oxygen permeability of 0.5×10⁹ cm³ cm/(s cm² cm Hg) or greater at 25° C., 1 atmosphere.

8. The cell culture article of claim 4, wherein the coating has an ultimate tensile modulus of less than 5 megapascals.

9. The cell culture article of claim 4, wherein the coating absorbs less than 25% of nefazodone in a phosphate buffered saline solution at a concentration of 200 micromolar after 24 hours of incubation on the cell culture surface.

10. The cell culture article of claim 4, wherein the substrate comprises polystyrene.

11. The cell culture article of claim 4, wherein the article is free of components of unknown origin.

12. A method comprising:
seeding cells on the cell culture surface of the article of claim 4, and contacting the seeded cells with cell culture medium.

13. The method of claim 12, wherein the cell culture surface is configured to cause the cells to exhibit an in vivo-like morphology.

14. The method of claim 13, wherein the cells are breast cells and wherein the cells form acinus structures when cultured on the cell culture surface.

15. A method for manufacturing a cell culture article comprising:
providing a substrate having a micro-structured surface; and
coating the micro-structured surface of the substrate with a thin layer of a polymer comprising polydimethylsiloxane to produce a micro-structured cell culture surface, wherein the layer is sufficiently thin such that the coating absorbs less than 25% of nefazodone in a phosphate buffered saline solution at a concentration of 200 micromolar after 24 hours of incubation on the cell culture surface.

16. The method of claim 15, wherein the polymer consists of polydimethylsiloxane.

17. A method for manufacturing a cell culture article comprising:
providing a substrate having a micro-structured surface; and
coating the micro-structured surface of the substrate with a thin layer of a polymer to produce a micro-structured cell culture surface, wherein the polymer has a water contact angle of greater than 20°, wherein the layer is sufficiently thin such that the coating absorbs less than 25% of nefazodone in a phosphate buffered saline solution at a concentration of 200 micromolar after 24 hours of incubation on the cell culture surface.

18. The method of claim 17, wherein the polymer layer has an ultimate tensile modulus of less than 5 megapascals.

19. The method of claim 17 wherein the polymer layer has an average thickness of between 5 micrometers and 100 micrometers.

20. The method of claim 17, wherein providing the substrate having the micro-structured surface comprises generating the substrate via molding.
